(19) United States
(12) Patent Application Publication
(10) Pub. No.: US 2025/0186681 A1
(43) Pub. Date: Jun. 12, 2025

(54) PACKAGING FOR DRUG DELIVERY DEVICE

(71) Applicant: AMGEN INC., Thousand Oaks, CA (US)

(72) Inventors: Kay Diehnelt, Glendale, CA (US); Edgardo Halili, Santa Clara, CA (US); Evan McDonnell, Thousand Oaks, CA (US); Lize Jaspers, Thousand Oaks, CA (US); Alberto David Herrera, Ventura, CA (US)

(21) Appl. No.: 18/845,286

(22) PCT Filed: Mar. 21, 2023
(86) PCT No.: PCT/US23/15733

§ 371 Date: Sep. 9, 2024

Related U.S. Application Data
(60) Provisional application No. 63/328,099, filed on Apr. 6, 2022.

(51) Int. Cl. A61M 5/00; B65B 5/06

(52) U.S. Cl. CPC A61M 5/002; B65B 5/06; A61M 2207/10; A61M 2209/06

(57) ABSTRACT

An intermediate bulk shipping container includes a box having an interior cavity and at least one tray including a body defining a plurality of recesses. Each of the plurality of recesses is dimensioned to accommodate a drug delivery device. The body of the at least one tray defines more than four recesses.

### CROSS-REFERENCE TO RELATED APPLICATION

Priority is claimed to U.S. Provisional Patent Application No. 63/328,099, filed Apr. 6, 2022, the entire contents of which are hereby incorporated by reference herein.

### FIELD OF DISCLOSURE

The present disclosure generally relates to drug delivery devices, and, more particularly, to packaging assemblies and/or configurations for drug delivery devices.

### BACKGROUND

Drug delivery devices, such as injectors, are used to deliver liquid drugs to a patient. Upon activation, a drug delivery device will expel a drug stored within an internal reservoir such as, for example, a pre-filled syringe (“PFS”) through a needle, cannula, or other delivery member into the patient. Some drug delivery devices, such as pen-type autoinjectors or on-body injectors, may be positioned adjacent to a patient's skin to deliver a drug via an injection needle or some other means over a period of time. The drug delivery device may be positioned near the tissue of the patient's abdomen, thigh, arm, or some other portion of the patient's body.

Oftentimes, such drug delivery devices are shipped to separate facilities that may perform additional processing steps (e.g., labelling), and/or end-user packaging. Manufacturers may use small shipper packages (e.g., packages containing four devices therein). Such intermediate packaging can create inefficiencies, as an individual must carefully physically remove adhesives or labels from the packaging and subsequently remove the device in order to perform the additional processing and/or repackaging steps. Such packaging is oftentimes discarded at this time, and may result in waste and excess packaging. Further, in some environments, some manufacturers may previously have assembled a number of devices and place them in bins with foam or other insulating material and place these containers in cold storage until ready for final processing. These devices were oftentimes susceptible to shifting while in this packaging and abrading against each other, which could lead to scratches and/or unintentional manual manipulation thereof. Further, such packaging may not be conducive to automated processes due to the devices not having a uniform orientation while disposed in these containers, and may lead to environmental and cost concerns.

The present disclosure sets forth packaging for drug delivery devices and corresponding approaches embodying advantageous alternatives to existing packaging and approaches, and that may address one or more of the challenges or needs mentioned herein.

### SUMMARY

In accordance with a first aspect, an intermediate bulk shipping container includes a box having an interior cavity and at least one tray including a body defining a plurality of recesses. Each of the plurality of recesses is dimensioned to accommodate a drug delivery device. The body of the at least one tray defines more than four recesses.

In some examples, the at least one tray may include a positioning notch formed on the body.

In some approaches, the at least one tray includes a plurality of trays. Each of the plurality of trays may be stackable such that the plurality of trays are disposable within the interior cavity of the box.

In these and other examples, a lid may be provided that covers the at least one tray. In some examples, this lid may include a lid positioning notch.

In some approaches, the lid may be constructed from an anti-static material.

In some approaches, a method of processing a plurality of drug delivery devices is provided that includes completing a first process step in which the plurality of drug delivery devices are at least partially assembled. An intermediate process step is completed using the previously-described intermediate bulk shipping container. A subsequent process step is completed in which the plurality of drug delivery devices are prepared for administration. In some examples, the intermediate process step may include transporting the plurality of drug delivery devices to a secondary location. Further, in some examples, the subsequent process step may include at least one of applying at least one label to each of the plurality of drug delivery devices or repackaging at least one of the drug delivery devices into an end-user container.

In accordance with an additional aspect, a drug processing system is provided that includes at least one tray. The at least one tray includes a body defining a plurality of recesses, each of the plurality of recesses dimensioned to accommodate a drug delivery device. The body further defines at least one sidewall at least partially surrounding each of the plurality of recesses. The at least one tray further includes a device removal facilitation mechanism at least partially formed on the at least one sidewall. The body of the at least one tray defines more than four recesses.

### BRIEF DESCRIPTION OF THE DRAWINGS

The above needs are at least partially met through provision of the packaging for drug delivery devices described in the following detailed description, particularly when studied in conjunction with the drawings, wherein:

FIG. 1 illustrates an example intermediate bulk shipping container in accordance with various embodiments;

FIG. 2 illustrates an example tray, drug delivery device, and lid for use with the example intermediate bulk shipping container of FIG. 1 in accordance with various embodiments;

FIG. 3 illustrates a cross-sectional perspective view of the example tray of FIG. 2 in accordance with various embodiments;

FIG. 4 illustrates a perspective view of a portion of the example tray of FIGS. 2 and 3 in accordance with various embodiments;

FIG. 5 illustrates a perspective view of an example drug processing system in accordance with various embodiments;

FIG. 6 illustrates a perspective view of an example lid for use with the example intermediate bulk shipping container of FIGS. 1-5 in accordance with various embodiments;

FIG. 7 illustrates the example tray of FIGS. 1-6 in a pre-shipment arrangement in accordance with various embodiments FIG. 8 illustrates the example tray of FIGS. 1-7 during packaging in accordance with various embodiments;

FIG. 9 illustrates the example tray of FIGS. 1-8 during packaging in accordance with various embodiments; and

FIG. 10 illustrates an alternative tray for use with an alternate drug delivery device design in accordance with various embodiments.

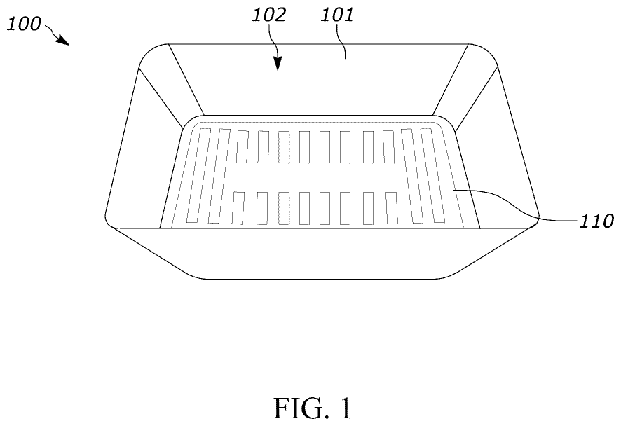

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various embodiments, bulk intermediate packaging for drug delivery devices is provided that safely transports these devices in an organized arrangement. The packaging described herein advantageously includes a large number of recesses (e.g., greater than four such as, for example, eight, 12, 16, 24, etc.) and may be stackable, reusable, and modular in nature. Further, such packaging allows a user and/or an automated or semi-automated process to quickly remove devices from the packaging with a single, continuous motion. Components of the packaging also ensure that each drug delivery device is properly placed therewithin, thus ensuring the devices will remain secure and may be removed quickly from the packaging.

The drug delivery device delivers a drug, which may also be referred to herein as a medicament or drug product. The drug may be, but is not limited to, various biologicals such as peptides, peptibodies, or antibodies. The drug may be in a fluid or liquid form, although the disclosure is not limited to a particular state. Various implementations and configurations of the drug delivery device are possible. For example, the present disclosure describes a drug delivery device in the form of a single-use, disposable injector. In other embodiments, the drug delivery device may be configured as multiple-use reusable injector. The drug delivery device may be operable for self-administration by a patient or for administration by a caregiver or a formally trained healthcare provider (e.g., a doctor or nurse). Further, the drug delivery device may take the form of an autoinjector or pen-type injector, and, as such, may be held in the hand of the user over the duration of drug delivery or dosing.

Turning now to the Figures, an intermediate bulk shipping container 100 is provided for use to ship complete (i.e., fully assembled) and/or partially complete (i.e., partially assembled) drug delivery devices 10. More specifically, the container 100 may include a box 101 having an interior cavity 102 and at least one tray 110. Further, in some examples and as will be discussed in further detail below, the container 100 may additionally include a lid 140 and an optional bag 150. The tray 110 includes a body 111 defining a number of recesses 114 that each accommodate and/or receive an individual drug delivery device 10. As illustrated in the Figures, in some examples, the tray 110 may include 16 recesses 114, but any number of recesses 114 may be provided. However, because the container 100 is used for bulk shipments, it is appreciated that the tray 110 may include greater than four recesses 114.

The body 111 of the tray 110 includes a first end 111a, a second end 111b, a first side 111c, and a second side 111d which cooperate to define an outer wall. Each of the recesses 114 are divided by sidewalls 116 that are shaped and dimensioned to correspond with the shape and size of the drug delivery device 10. The tray 110 further includes a device removal facilitation mechanism 118 in the form of a groove and/or internal contoured cavity that extends a length of the body 111 of the tray 110. More specifically, the groove 118 extends through each sidewall 116, and, optionally, also extends a length below the recess 114. Further, the groove 118 also extends onto the sidewall 116 of the tray 111 formed at the first and second sides 1110, 111d. In some examples, the groove 118 may be dimensioned to receive a finger or other grasping and/or removal mechanism in implementations where automated and/or semi-automated processes are provided. Notably, the groove 118 facilitates a cascading and/or swiping removal of the devices 10, and eliminates a need to remove each individual device 10 via an individualized plucking or grasping motion.

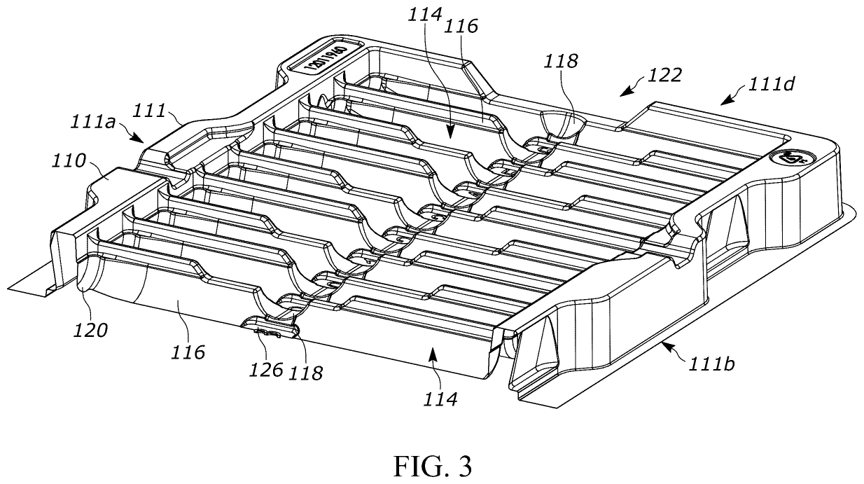

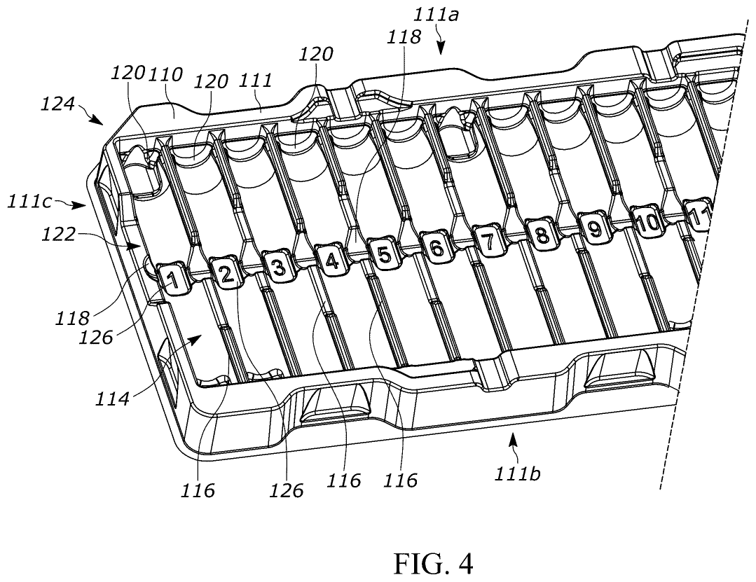

With reference to FIGS. 3 and 4, the tray 110 may further include an orientation mechanism 120 disposed in or near each recess 114. In the illustrated example, the orientation mechanism 120 is disposed near the first end 111a of the tray 111, but other alternative locations are possible. In the illustrated examples, the orientation mechanism 120 is in the form of an undercut region that receives a portion of the device 10. This undercut region may be shaped and sized such that only a particular end of the device 10 (e.g., the “top” end) may fit therewithin, meaning if the device 10 is placed in the recess 114 in the wrong or opposite orientation (e.g., the “bottom” end being disposed therewithin), the device 10 is prevented from fully nesting within the recess 114. Notably, the tray 110 may also include inspection notches 122 positioned along the first and second sides 111c, 111d. These notches 122 may allow a user to visually inspect the devices 10 to ensure they are fully seated within their respective recess 114. Upon placing the devices 10 within the recesses 114, a user may view the inspection notches 122 from the side to determine whether any devices are protruding from their respective recess 114. In some examples, a user may manually inspect the tray 110, and in other examples, the inspection process may be automated using an imaging system and/or a computing system. Other examples are possible.

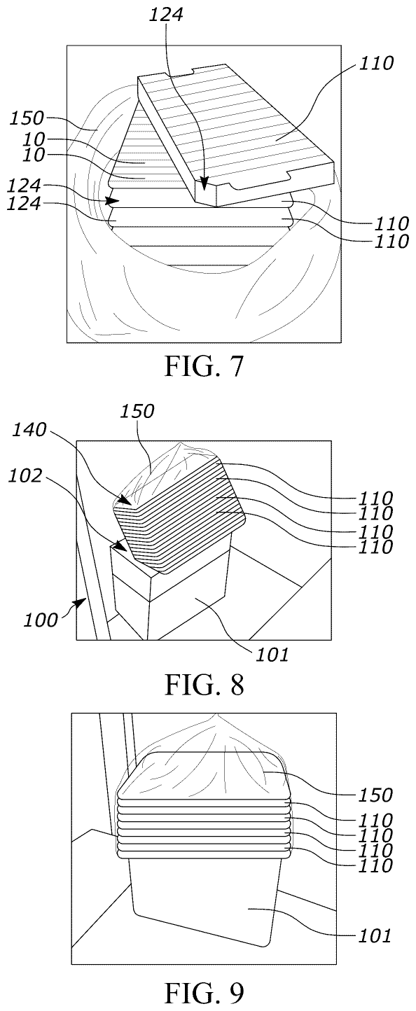

In some examples, the tray 110 may be constructed from a reusable material (e.g., a polymeric material). Further, in some examples, the tray 110 may be constructed from a material having anti-static characteristics, thus reducing the likelihood of unwanted debris adhering to the drug delivery device 10. The tray 110 may be rigid or semi-rigid, and may support additional trays in a nesting or stacked configuration (see, e.g., FIGS. 7-9). Accordingly, the trays 110 may be used in a modular fashion to receive any desired number of devices 10 by simply stacking additional trays 110 on top of each other.

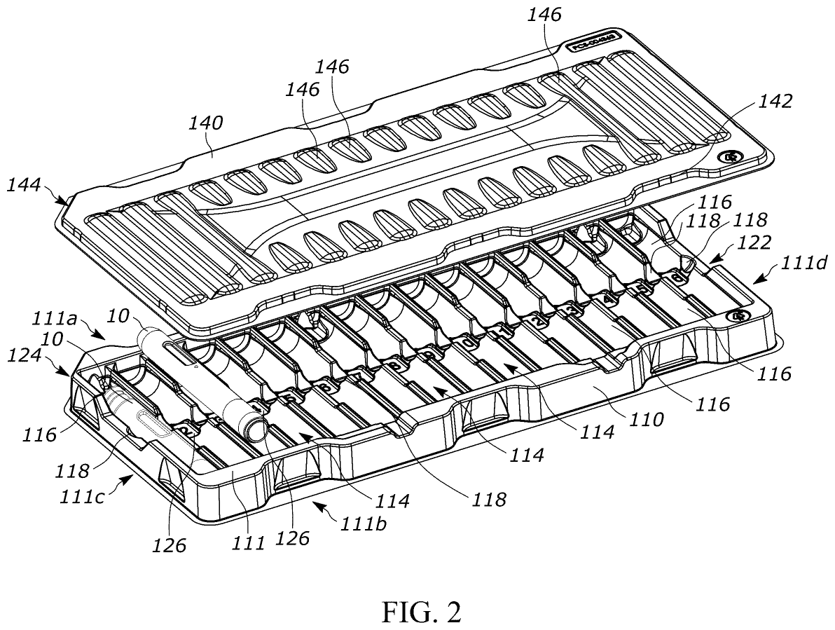

With reference to FIGS. 2 and 4, the tray 110 may further include a positioning notch 124. In the illustrated example, the positioning notch 124 is in the form of a cutout between the first end 111a and the first side 111c of the tray, but other locations are possible. The positioning notch 124 may be used to ensure the tray 110 itself is properly oriented relative to other components such as, for example, other trays 110 and/or other processing components. More specifically, if a number of trays are stacked on top of each other, a user (or automated or semi-automated process) may quickly identify whether a tray 110 is oriented properly by determining whether the positioning notch 124 is present at a corner when viewed from a top or elevation view. If a portion of the body 111 of the tray 110 is visible, a user may quickly rotate the tray 110 in the correct orientation prior to performing additional processing steps. As a result, a user may ensure that both the trays 110 and the devices 10 disposed thereon are all properly oriented.

With continued reference to FIGS. 2 and 4, the trays 110 further include a visual identifier 126 formed in or along each recess 114. In the illustrated example, the visual identifier 126 is in the form of successive numerals which may be viewed by a user and/or imaging system. These visual identifiers 126 may allow the user and/or imaging system to readily identify recesses 114 that require attention (e.g., if a device 10 is missing and/or properly oriented). Other examples are possible.

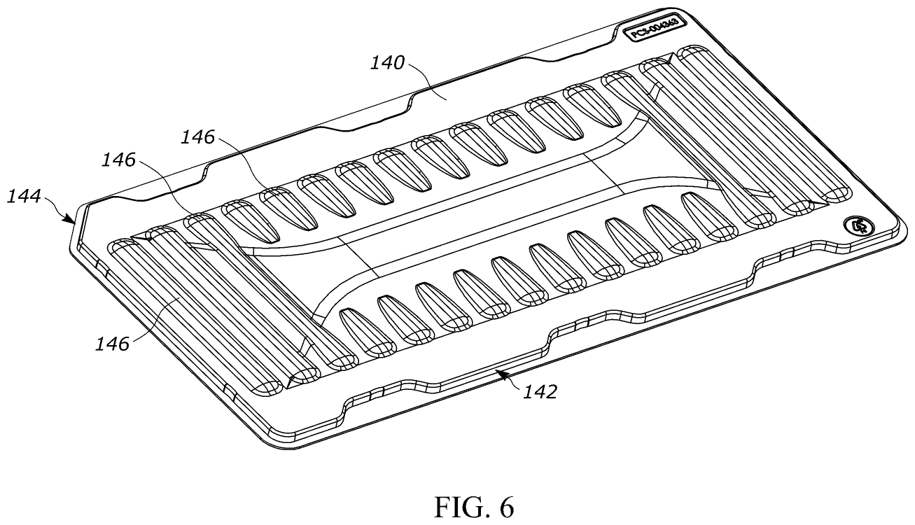

With reference to FIGS. 2 and 6, the lid 140 may be used to cover the tray 110. The lid 140 is dimensioned to be placed on top of the tray 110, and may include recesses on an underside thereof (not illustrated) that correspond to the shape of the device 10. Further, an upper side 140a of the lid 140 includes an outer ledge 142 as well as protrusions 146. The outer ledge 142 may have a geometry that corresponds with a lower surface of the tray 110 such that the outer ledge 142 may next within a portion of the tray 110 when the lid 140 is used and trays 110 are stacked on top of each other. Further, the protrusions 146 are positioned and dimensioned to engage corresponding cavities formed on the underside of the tray 110. So arranged, the tray may nest within and/or otherwise be coupled with a tray 110 stacked above. In some examples, the tray 110 may be releasably secured with the lid 140 via any number of suitable approaches such as, for example, a friction-fit coupling.

In some examples, the lid 140 further includes a lid positioning notch 144 along the outer edge 142. In these examples, the lid positioning notch 144 may have a similar shape and/or dimension as the positioning notch 124 of the tray 110. As such, when the lid or lids 110 are placed on the tray or trays 110, a user may still be able to quickly identify whether any trays 110 or lids 140 are incorrectly oriented by viewing the trays 110 from above. Other examples are possible.

As illustrated in FIGS. 7-9, a bag 150 may be sized to receive any number of trays 110 and/or lids 140. The bag 150 allows the tray or trays 110 to quickly be moved into or out of the box 101. As stated, the bag 150 is optional. The bag 150 is optional because the disclosed configuration of the lid positioning notch 144 and positioning notch 124 of the tray 110 stabilize the load such that the bag 150 is not necessary. As illustrated in FIGS. 8 and 9, the trays 110, lids 140, and bag 150 may be placed within the box, and a user may visually inspect the arrangement to ensure the positioning notch 124 is properly oriented in each of the trays 110 and lids 140.

In use, the trays 110 may be provided at a manufacturing and/or assembly site that processes a number of drug delivery devices 10. In such environments, a first process step may commence where the drug delivery devices 10 are partially and/or completely assembled. The desired number of devices 10 may then be placed into the recesses 144 of the tray or trays 110, whereupon an intermediate process step may occur. In some examples, this intermediate process step may include transporting the tray or trays 110 (and any additional packaging such as the box 101 and/or the optional bag 150) to a different location. More specifically, the tray or trays 110 may be transported to a storage facility until additional processing is desired, the tray or trays 110 may be transported to a different location within the manufacturing and/or assembly site for additional processing, and/or the tray or trays 110 may be transported to a different facility such as, for example, a labeling facility, a laboratory, a healthcare facility, etc. As such, the tray or trays 110 may be used during intermediate processing steps to facilitate the transport of bulk quantities of devices 10.

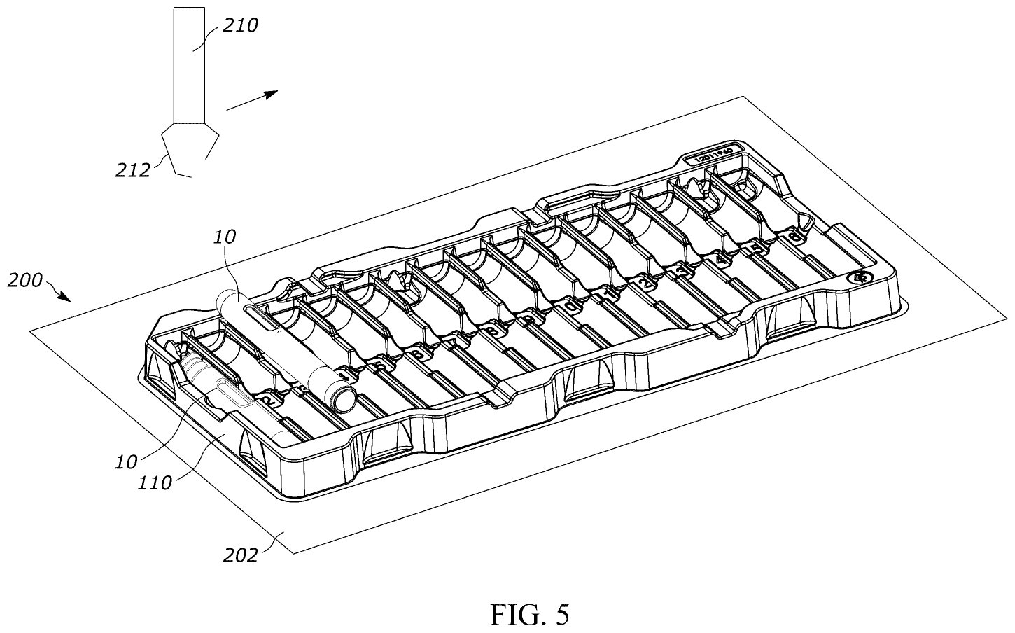

In some examples, the method may further include completion of a subsequent processing step. For example, after the tray or trays 110 are transported, a final step such as labeling each device 10, repackaging the devices 10 into a final (e.g., end-user) packaging, etc., may occur. As previously noted, in examples where the devices 10 are to be removed from the tray 110, after removing the tray or trays 110 from the optional bag 150 and removing any lids 140 therefrom, a user may quickly remove the devices in a single, continuous motion. In some examples, and as illustrated in FIG. 5, the subsequent processing step may be at least partially performed by a drug processing system 200. The system 200 may have automated and/or semi-automated processing capabilities to reduce overall processing times. In these examples, the system 200 may include a platform 202 that receives and/or supports the tray 110 and an arm 210. In some arrangements, the platform 202 may be in the form of a conveyor belt, a stationary table, a workstation, and the like, and may include a recess (not illustrated) or other positioning mechanism that receives and positions the tray 110 at a desired location on the platform 202. As an example, the platform 202 may include a recess having an angled portion that corresponds with the positioning notch 124 formed on the tray 110 to ensure the tray 110 is correctly positioned.

The arm 210 may be positioned above the tray 110 while the tray 110 is disposed on the platform 202. In some examples, the arm 210 may include a telescoping member to selectively raise and/or lower a grasping mechanism 212. More specifically, the grasping mechanism 212 may be in the form of finger-like members having a curvature capable of engaging the recess 114 formed on the tray 110. By properly orienting the tray 110 on the platform 202, the arm 210 may be programmed to lower the grasping mechanism 212 into the recess 114 and complete a swiping motion to remove all of the devices 10 disposed on the tray 110, whereupon the devices 10 may be collected and used as desired. Advantageously, because the tray 110, and particularly the orientation mechanism 120, ensures the devices 10 are all oriented in the same direction, a user and/or the system needn't turn or flip devices 10 over in order to properly complete additional processing steps such as labelling (which may require the label to be adhered to the device 10 at a specific location and orientation) and/or subsequent packaging (which may require the device 10 to be disposed in a particular arrangement). As such, the overall time needed to process these devices 10 may be reduced, and may result in significant cost savings.

As compared with conventional, single-use, non-bulk packaging, the presently-described packaging advantageously allows a user or automated process the ability to remove all of the devices therefrom in a single swiping and/or cascading motion as opposed to the previously-used individualized scooping motion. Such a swiping and/or cascading motion results in substantially reduced processing times, and allows for the incorporation of automated processing systems which previously were not feasible. Such packaging may also be used for storage and/or after any intermediate processing steps are completed. More specifically, the packaging may be used for applications such as clinical trials, shipments to distributors, and/or foreign markets. In such examples, overall costs may be reduced due to reuse of the packaging.

Further, the present container 100 advantageously incorporates stackable trays that are modular in nature. Any number of trays may be stacked into the shipping box to ship the desired number of drug delivery devices. Further, such trays may be used as a storage device even after additional processing steps have occurred, thereby reducing the need for additional packaging and/or shipping materials. The lid may be easily removed from the tray, and, like, the trays themselves, may allow a user to quickly identify whether the trays and the lid are oriented in a similar direction prior to placing them in the shipping/transporting box or other packaging. So arranged, this visual identifier ensures the proper orientation when performing automated (or semi-automated) processing steps where relative alignments of components is important.

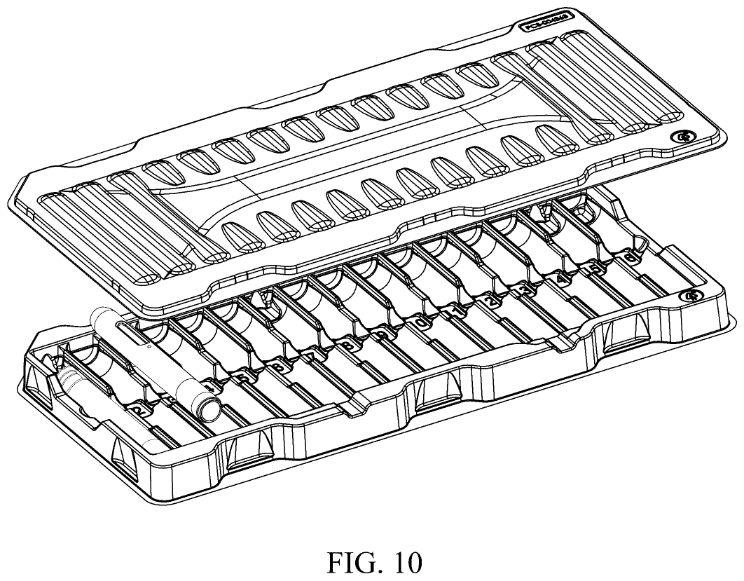

Additionally, as illustrated in FIG. 10, the packaging described herein may be shaped to be used with any number of different drug delivery devices having varying sizes, shapes, and/or configurations.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF: cMet axis (HGF/SF: c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-a4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFa monoclonal antibody); Reopro® (abciximab, anti-GP Ilb/llia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFa mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-a531 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNa mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGß mAb (MDX-1307); anti-mesothelin dsFv-PE38 conjugate (CAT-5001); anti-PD1mAb (MDX-1106 (ONO-4538)); anti-PDGFRa antibody (IMC-3G3); anti-TGFß mAb (GC-1008); anti-TRAIL Receptor-2 human mAb (HGS-ETR2); anti-TWEAK mAb; anti-VEGFR/FIt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG12C small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein (a), also known as Lp (a), or another product containing a small interfering RNA (siRNA) that lowers lipoprotein (a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. An intermediate bulk shipping container including:
a box having an interior cavity;
at least one tray including a body defining a plurality of recesses, each of the plurality of recesses dimensioned to accommodate a drug delivery device;
wherein the body of the at least one tray defines more than four recesses.

2. The intermediate bulk shipping container of claim 1, wherein the at least one tray includes a positioning notch formed on the body.

3. The intermediate bulk shipping container of claim 1, wherein the at least one tray comprises a plurality of trays, wherein each of the plurality trays is stackable such that the plurality of trays are disposable within the interior cavity of the box.

4. The intermediate bulk shipping container of claim 1, further comprising a lid adapted to cover the at least one tray.

5. The intermediate bulk shipping container of claim 4, wherein the lid includes a lid positioning notch.

6. The intermediate bulk shipping container of claim 1, wherein the at least one tray is constructed from an anti-static material.

7. A method of processing a plurality of drug delivery devices comprising:
completing a first process step in which the plurality of drug delivery devices are at least partially assembled;
completing an intermediate process step using the intermediate bulk shipping container of any one of claims 1-6; and
completing a subsequent process step in which the plurality of drug delivery devices are prepared for administration.

8. The method of claim 7, wherein the intermediate process step includes transporting the plurality of drug delivery devices to a secondary location.

9. The method of claim 7, wherein the subsequent process step includes at least one of applying at least one label to each of the plurality of drug delivery devices or repackaging at least one of the drug delivery devices into an end-user container.

10. A drug processing system comprising:
at least one tray including:
a body defining a plurality of recesses, each of the plurality of recesses dimensioned to accommodate a drug delivery device, the body further defining at least one sidewall at least partially surrounding each of the plurality of recesses, and
a device removal facilitation mechanism at least partially formed on the at least one sidewall,
wherein the body of the at least one tray defines more than four recesses.

11. The drug processing system of claim 10, wherein the device removal facilitation mechanism includes a groove extending along a length of the body of the tray.

12. The drug processing system of claim 10, further comprising an orientation mechanism disposed in or near each of the plurality of recesses, the orientation mechanism adapted to orient each of the drug delivery devices in a first position.

13. The drug processing system of claim 12, wherein the orientation mechanism includes an undercut portion.

14. The drug processing system of claim 10, further comprising an arm positioned above the at least one tray, the arm including a grasping mechanism adapted to remove each of the drug delivery devices disposed within the plurality of recesses in a continuous motion.

15. The drug processing system of claim 14, further comprising a platform adapted to support the at least one tray.

16. The drug processing system of claim 15, wherein the platform is further adapted to transport the at least one tray to a subsequent processing station.

17. The drug processing system of claim 10, further comprising a visual identifier disposed within each of the plurality of recesses.

18. The drug processing system of claim 10, further comprising a positioning notch formed on the body.

19. The drug processing system of claim 10, wherein the at least one tray is reusable.
